SEQUENCE LISTING (4 sequences)

SEQ ID NO: 1 - DNA, 1000 bp, Corynebacterium glutamicum
  CDS (186)..(815)
gcacggcaaaataagtcataacaggtaagtttaggcgtctagacgtacactaaacggaatttggttcacttatattcttctcttgcttttgtggcgcttcgtccgtcgagaggtttttaggaatagagtgggctcaagctttgtgacaagttttttggagaaatcattactagtcgtagtcttcaatttgggtgcaggtagggtggaacaccgtgagtgatgtatcagcaggcgtaaatggcgcacaagatccaagcaatcaagcggtcaagccttccaactggaaccttccgaacttcttgaccagcttgcgtatcattgtcatccctttgtttgcgtggcttacgcttaaaggtgagacggaaaacaatgcttttgcctggtgggcgttggttgttttcattttgctcatgatcaccgacaagcttgacggcgatattgcgcgagcacgtggcctggtcactgactttggcaagatcgcggatccgattgccgataaggcgttgatgaccacagcatttgtctgtttcaacatcatcggcattttgccctggtgggtcactgcgttgattgtgcttcgagagttcggcattaccatctggcgtttcttccaactgcgcgctggaaatgttgtgcctgcatcaaaggggggcaagcttaagactgctctgcagactgttgccgttgctctgtatctgtgccctttcccaagttggatggatattccaagccagatcgtcatgtatgcagcgctgatcgtcaccgtggtcacgggtctgcagtacctgtgggattcacgaaagtccgcagaaagctagaccatgtcggagaatctggcggggcgagtggtggagctgttgaaatcgcgcggtgaaacgctggcgttttgtgaatccctcaccgccggccttgccagtgcgacgatcgcagagatccccggcgcctcagtggtacttaaaggcgggctggtcacctatgccaccgagcttaaggttgcgct

SEQ ID NO: 2 - protein, 210 aa, Corynebacterium glutamicum
IWVQVGWNTVSDVSAGVNGAQDPSNQAVKPSNWNLPNFLTSLRIIVIPLFAWLTLKGETENNAFAWWALVVFILLMITDKLDGDIARARGLVTDFGKIADPIADKALMTTAFVCFNIIGILPWWVTALIVLREFGITIWRFFQLRAGNVVPASKGGKLKTALQTVAVALYLCPFPSWMDIPSQIVMYAALIVTVVTGLQYLWDSRKSAES

SEQ ID NO: 3 - DNA, 31 bp, Corynebacterium glutamicum
tgctctagacgtccgtcgagaggtttttagg

SEQ ID NO: 4 - DNA, 30 bp, Corynebacterium glutamicum
tgctctagaccccgccagattctccgacat

(19) United States
(12) Patent Application Publication
(10) Pub. No.: US 2002/0155555 A1
(43) Pub. Date: Oct. 24, 2002

(54) Nucleotide sequences which code for the pgsA2 gene

(76) Inventors: Madhavan Nampoothiri K., Kerala (IN); Bettina Mockel, Dusseldorf (DE); Walter Pfefferle, Halle (DE); Lothar Eggeling, Julich (DE); Hermann Sahm, Julich (DE)

Correspondence Address: SMITH, GAMBRELL & RUSSELL, LLP, Suite 800, 1850 M Street, N.W., Washington, DC, 20036, US

(21) Appl. No.: 09/855,835

(22) Filed: May 16, 2001

Related U.S. Application Data
(63) Continuation-in-part of application No. 09/577,855, filed on May 25, 2000, now abandoned.

(30) Foreign Application Priority Data
May 4, 2000 (DE) 100 21 829.6

(51) Int. Cl. C12P 13/08; A01N 63/00; A61K 31/70; A01N 43/04

(52) U.S. Cl. 435/115; 435/106; 424/93.4; 514/44

(57) ABSTRACT

The invention relates to a genetically modified coryneform bacterium, the pgsA2 gene of which is amplified, and an isolated polynucleotide which codes for CDP-diacylglycerol-glycerol-3-phosphate 3-phosphatidyltransferase from coryneform bacteria, and also a method for the fermentative preparation of L-amino acids with amplification of the pgsA2 gene in the bacteria and the use of the polynucleotide as a primer or hydridization probe.

### RELATED APPLICATION DATA

This application is a Continuation-In-Part of co-pending U.S. patent appln. Ser. No. 09/577,855 filed May 25, 2000, which application claims priority under 35 U.S.C.§119 from German Patent Appln. No. 10021829.6, filed in Germany on May 4, 2000. The above-identified U.S. patent application and German patent application are entirely incorporated herein by reference. The invention provides genetically modified coryneform bacteria, nucleotide sequences which code for CDP-diacylglycerol-glycerol-3-phosphate 3-phosphatidyltransferase and methods for the fermentative preparation of amino acids, in particular L-lysine, using coryneform bacteria in which the pgsA2 gene, which codes for CDP-diacylglycerol-glycerol-3-phosphate 3-phosphatidyltransferase (EC 2.7.8.5), is amplified. All references cited herein are expressly incorporated by reference. Incorporation by reference is also designated by the term “I.B.R.” following any citation.

### BACKGROUND ART

Amino acids, in particular L-lysine, are used in human medicine and in the pharmaceuticals industry, but in particular in animal nutrition.

It is known that amino acids are prepared by fermentation from strains of coryneform bacteria, in particular Corynebacterium glutamicum. Because of their great importance, work is constantly being undertaken to improve the preparation methods. Improvements to the methods can relate to fermentation measures, such as e. g. stirring and supply of oxygen, or the composition of the nutrient media, such as e. g. the sugar concentration during the fermentation, or the working up to the product form by e. g. ion exchange chromatography, or the intrinsic output properties of the microorganism itself.

Methods of mutagenesis, selection and mutant selection are used to improve the output properties of these microorganisms. Strains which are resistant to antimetabolites, such as e. g. the lysine analogue S-(2-aminoethyl)-cysteine, or are auxotrophic for metabolites of regulatory importance and produce L-amino acids, such as e. g. L-lysine, are obtained in this manner.

Methods of the recombinant DNA technique have furthermore been employed for some years for improving the strain of Corynebacterium strains which produce amino acids, by amplifying individual amino acid biosynthesis genes and investigating the effect on the amino acid production. Review articles in this context are to be found, inter alia, in Kinoshita (“Glutamic Acid Bacteria”, in: Biology of Industrial Microorganisms, Demain and Solomon (Eds.), Benjamin Cummings, London, UK, 1985, 115-142 I.B.R.) Hilliger (BioTec 2, 40-44 (1991) I.B.R.), Eggeling (Amino Acids 6:261-272 (1994) I.B.R.), Jetten and Sinskey (Critical Reviews in Biotechnology 15, 73-103 (1995) I.B.R.) and Sahm et al. (Annuals of the New York Academy of Science 782, 25-39 (1996) I.B.R.).

### OBJECT OF THE INVENTION

The object of the present invention was to provide new aids for improved fermentative preparation of amino acids, in particular L-lysine.

This object is achieved by a genetically modified coryneform bacterium, the pgsA2 gene of which, which codes for CDP-diacylglycerol-glycerol-3-phosphate 3-phosphatidyltransferase, is amplified.

Amino acids, in particular L-lysine, are used in human medicine, in the pharmaceuticals industry and in particular in animal nutrition. There is therefore a general interest in providing new improved methods for the preparation of amino acids, in particular L-lysine.

When L-lysine or lysine are mentioned in the following, not only the base but also the salts, such as e. g. lysine monohydrochloride or lysine sulfate, are also meant by this.

### SUMMARY OF THE INVENTION

The new DNA sequence of C. glutamicum which codes for the cma gene and which as a constituent of the present invention is SEQ ID NO 1 and related sequences. The amino acid sequence of the corresponding gene product of the pgsA2 gene has furthermore been derived from the present DNA sequence. The resulting amino acid sequence of the pgsA2 gene product is SEQ ID NO 2 and related sequences.

### BRIEF DESCRIPTION OF THE DRAWINGS

The present invention will be further understood with reference to the drawing offered here for illustration only and not in limitation of this invention.

FIG. 1: Plasmid map of pJC1pgsA2

### DETAILED DESCRIPTION OF THE INVENTION

The invention provides a genetically modified coryneform bacterium, in which its pgsA2 gene, which codes for CDP-diacylglycerol-glycerol-3-phosphate 3-phosphatidyltransferase, is amplified.

The term “amplification” in this connection describes the increase in intracellular activity of one or more enzymes in a microorganism which are coded by the corresponding DNA.

Amplification can be achieved with the aid of various manipulations of the bacterial cell.

To achieve an amplification, in particular an over-expression, the number of copies of the corresponding genes can be increased, a potent promoter can be used, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative L-lysine production. It is also possible to use a gene which codes for a corresponding enzyme with a high activity. The expression is likewise improved by measures to prolong the life of the m-RNA. Furthermore, the enzyme activity is also increased overall by preventing the degradation of the enzyme. These measures can optionally also be combined as desired.

The microorganisms which the present invention provides can prepare L-amino acids, in particular L-lysine, from glucose, sucrose, lactose, fructose, maltose, molasses, starch, cellulose or from glycerol and ethanol. They can be representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum, are, for example, the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

and L-lysine-producing mutants or strains prepared therefrom, such as, for example

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464 and

Corynebacterium glutamicum DSM5715.

The present invention also provides an isolated polynucleotide from coryneform bacteria, comprising a polynucleotide sequence chosen from the group consisting of

a) polynucleotide which is homologous to the extent of at least 70% with a polynucleotide which codes for a polypeptide which comprises the amino acid sequence of SEQ ID No. 2,

b) polynucleotide which codes for a polypeptide which comprises an amino acid sequence which is homologous to the extent of at least 70% with the amino acid sequence of SEQ ID No. 2,

c) polynucleotide which is complementary to the polynucleotides of a) or b), and

d) polynucleotide comprising at least 15 successive nucleotides of the polynucleotide sequence of a), b) or c).

In the context of the present Application, a polynucleotide sequence is “homologous” to the sequence according to the invention if it coincides in its base composition and sequence with the sequence according to the invention to the extent of at least 70%, preferably at least 80%, particularly preferably at least 90%. According to the present invention, a “homologous protein” is to be understood as proteins which have an amino acid sequence which coincides with the amino acid sequence coded by the pgsA2 gene (SEQ ID No.1) to the extent of at least 70%, preferably at least 80%, particularly preferably at least 90%, “coincide” being understood as meaning that either the corresponding amino acids are identical or they are amino acids which are homologous to one another. Those amino acids which correspond in their properties, in particular in respect of charge, hydrophobicity, steric properties etc., are called “homologous amino acids”.

The invention also provides a polynucleotide as described above , this preferably being a DNA which is capable of replication, comprising:

(i) the nucleotide sequence shown in SEQ ID no. 1, or

(ii) at least one sequence which corresponds to sequence (i) in the context of the degeneration of the genetic code, or

(iii) at least one sequence which hybridizes with the sequence complementary to sequence (i) or (ii), and optionally

(iv) mutations of neutral function in (i) which lead to the same or a homologous amino acid.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R. Hybridization of complementary sequences can occur at varying degrees of stringency. Sambrook et al.: Molecular Cloning, A Laboratory Manual (Cold Spring Harbor Laboratory Press, 1989) I.B.R.

Hybridization of complementary sequences can occur at varying degrees of stringency. Sambrook et al.: Molecular Cloning, A Laboratory Manual (Cold Spring Harbor Laboratory Press, 1989) I.B.R. Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R.

Comprehensive descriptions can be found in known textbooks of genetics and molecular biology, such as e. g. that by Hagemann (“Allgemeine Genetik” [General Genetics], Gustav Fischer Verlag, Stuttgart, 1986) I.B.R.

Possible mutations are transitions, transversions, insertions and deletions. Depending on the effect of the amino acid exchange on the enzyme activity, missense mutations or nonsense mutations are referred to. Insertions or deletions of at least one base pair in a gene lead to frame shift mutations, as a consequence of which incorrect amino acids are incorporated or translation is interrupted prematurely. Deletions of several codons typically lead to a complete loss of the enzyme activity.

Instructions on generation of such mutations are prior art and can be found in known textbooks of genetics and molecular biology, such as e. g. the textbook by Knippers (“Molekulare Genetik” [Molecular Genetics], 6th edition, Georg Thieme Verlag, Stuttgart, Germany, 1995) I.B.R., that by Winnacker (“Gene und Klone” [Genes and Clones], VCH Verlagsgesellschaft, Weinheim, Germany, 1990) I.B.R. or that by Hagemann (“Allgemeine Genetik” [General Genetics], Gustav Fischer Verlag, Stuttgart, 1986) I.B.R.

The invention also provides a vector containing one of the stated polynucleotides and coryneform bacteria acting as host cell which contain the vector or in which the pgsA2 gene is amplified.

The invention also provides

a preferably recombinant polynucleotide which is capable of replication in coryneform bacteria and comprises the nucleotide sequence SEQ ID No. 1,

a polynucleotide which codes for a polypeptide which comprises the amino acid sequence SEQ ID No. 2,

a vector containing the DNA sequence of C. glutamicum which codes for the pgsA2 gene, contained in the vector pJC1pgsA2, deposited in Corynebacterium glutamicum under number 13251,

and coryneform bacteria serving as the host cell, which contain the vector or in which the pgsA2 gene is amplified.

The invention also provides polynucleotides which comprise the complete gene with the polynucleotide sequence corresponding to SEQ ID No. 1 or fragments thereof, and which are obtainable by screening by means of hybridization of a corresponding gene library with a probe which comprises the sequence of the polynucleotide mentioned, according to SEQ ID No. 1, or a fragment thereof, and isolation of the DNA sequence mentioned.

Polynucleotide sequences according to the invention are also suitable as hybridization probes for RNA, cDNA and DNA, in order to isolate, in the full length, cDNA which code for CDP-diacylglycerol-glycerol-3-phosphate 3-phosphatidyltransferase and to isolate those cDNA or genes which have a high similarity of with the sequence of the CDP-diacylglycerol-glycerol-3-phosphate 3-phosphatidyltransferase gene.

Polynucleotide sequences according to the invention are furthermore suitable as primers for the polymerase chain reaction (PCR), for the preparation of DNA which codes for CDP-diacylglycerol-glycerol-3-phosphate 3-phosphatidyltransferase proteins.

Such oligonucleotides which serve as probes or primers can comprise more than 30, preferably up to 30, particularly preferably up to 20, very particularly preferably at least 15 successive nucleotides. Oligonucleotides which have a length of at least 40 or 50 nucleotides are also suitable.

“Isolated” means separated out of its natural environment.

“Polynucleotide” in general relates to polyribonucleotides and polydeoxyribonucleotides, it being possible for these to be non-modified RNA or DNA or modified RNA or DNA.

“Polypeptides” is understood as meaning peptides or proteins which comprise two or more amino acids bonded via peptide bonds.

The polypeptides according to the invention include a polypeptide according to SEQ ID No. 2, in particular those with the biological activity of the CDP-diacylglycerol-glycerol-3-phosphate 3-phosphatidyltransferase protein, and also those which are homologous to the extent of at least 70% with the polypeptide according to SEQ ID No. 2, and preferably are homologous to the extent of at least 80% and in particular to the extent of at least 90% to 95% with the polypeptide according to SEQ ID no. 2, and have the activity mentioned.

The invention moreover provides a method for the fermentative preparation of amino acids, in particular L-lysine, using coryneform bacteria which in particular already produce an amino acid, and in which the nucleotide sequences which code for the pgsA2 gene are amplified, in particular over-expressed.

The pgsA2 gene of C. glutamicum which codes for CDP-diacylglycerol-glycerol-3-phosphate 3-phosphatidyltransferase is described for the first time in the present invention.

To isolate the pgsA2 gene or also other genes of C. glutamicum, a gene library of this microorganism is first set up in E. coli. The setting up of gene libraries is described in generally known textbooks and handbooks. The textbook by Winnacker: Gene und Klone, Eine Einführung in die Gentechnologie [Genes and Clones, An Introduction to Genetic Engineering] (Verlag Chemie, Weinheim, Germany, 1990 I.B.R.) or the handbook by Sambrook et al.: Molecular Cloning, A Laboratory Manual (Cold Spring Harbor Laboratory Press, 1989 I.B.R.) may be mentioned as an example. A well-known gene library is that of the E. coli K-12 strain W3110 set up in λ vectors by Kohara et al. (Cell 50, 495 -508 (1987) I.B.R.). Bathe et al. (Molecular and General Genetics, 252:255-265, 1996 I.B.R.) describe a gene library of C. glutamicum ATCC13032, which was set up with the aid of the cosmid vector SuperCos I (Wahl et al., 1987, Proceedings of the National Academy of Sciences USA, 84:2160-2164 I.B.R.) in the E. coli K-12 strain NM554 (Raleigh et al., 1988, Nucleic Acids Research 16:1563-1575 I.B.R.). Börmann et al. (Molecular Microbiology 6(3), 317-326)) (1992) I.B.R.) in turn describe a gene library of C. glutamicum ATCC13032 using the cosmid pHC79 (Hohn and Collins, Gene 11, 291-298 (1980) I.B.R.). To prepare a gene library of C. glutamicum in E. coli it is also possible to use plasmids such as pBR322 (Bolivar, Life Sciences, 25, 807-818 (1979) I.B.R.) or pUC9 (Vieira et al., 1982, Gene, 19:259-268 I.B.R.). Suitable hosts are, in particular, those E. coli strains which are restriction- and recombination-defective. An example of these is the strain DH5αmcr, which has been described by Grant et al. (Proceedings of the National Academy of Sciences USA, 87 (1990) 4645-4649 I.B.R.). The long DNA fragments cloned with the aid of cosmids can then in turn be subcloned and subsequently sequenced in the usual vectors which are suitable for sequencing, such as is described e. g. by Sanger et al. (Proceedings of the National Academy of Sciences of the United States of America, 74:5463-5467, 1977 I.B.R.).

The new DNA sequence of C. glutamicum which codes for the pgsA2 gene and which, as SEQ ID No. 1, is a constituent of the present invention, was obtained in this manner. The amino acid sequence of the corresponding protein has moreover been derived from the present DNA sequence by the methods described above. The resulting amino acid sequence of the pgsA2 gene product is shown in SEQ ID No. 2.

Coding DNA sequences which result from SEQ ID No. 1 by the degeneracy of the genetic code are also a constituent of the invention. In the same way, DNA sequences which hybridize with SEQ ID No. 1 or parts of SEQ ID No. 1 are a constituent of the invention. Conservative amino acid exchanges, such as e. g. exchange of glycine for alanine or of aspartic acid for glutamic acid in proteins, are moreover known among experts as “sense mutations” which do not lead to a fundamental change in the activity of the protein, i.e. are of neutral function. It is moreover known that changes on the N and/or C terminus of a protein cannot substantially impair the function thereof or can even stabilize this. Information in this context can be found by the expert, inter alia, in Ben-Bassat et al. (Journal of Bacteriology 169:751-757 (1987) I.B.R.), in O'Regan et al. (Gene 77:237-251 (1989)), in Sahin-Toth et al. (Protein Sciences 3:240-247 (1994) I.B.R.), in Hochuli et al. (Bio/Technology 6:1321-1325 (1988) I.B.R.) and in known textbooks of genetics and molecular biology. Amino acid sequences which result in a corresponding manner from SEQ ID No. 2 are also a constituent of the invention.

In the same way, DNA sequences which hybridize with SEQ ID No. 1 or parts of SEQ ID No. 1 are a constituent of the invention. Finally, DNA sequences which are prepared by the polymerase chain reaction (PCR) using primers which result from SEQ ID NO. 1 are a constituent of the invention. Such oligonucleotides typically have a length of at least 15 nucleotides.

Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R.. Instructions for amplification of DNA sequences with the aid of the polymerase chain reaction (PCR) can be found by the expert, inter alia, in the handbook by Gait: Oligonukleotide synthesis: a practical approach (IRL Press, Oxford, UK, 1984 I.B.R.) and in Newton and Graham: PCR (Spektrum Akademischer Verlag, Heidelberg, Germany, 1994) I.B.R.

In the work on the present invention, it has been found that coryneform bacteria produce amino acids, in particular L-lysine, in an improved manner after amplification of the pgsA2 gene.

The genes or gene constructs under consideration can either be present in plasmids with a varying number of copies, or can be integrated and amplified in the chromosome. Alternatively, an over-expression of the genes in question can moreover be achieved by changing the composition of the media and the culture procedure.

Instructions in this context can be found by the expert, inter alia, in Martin et al. (Bio/Technology 5, 137-146 (1987) I.B.R.), in Guerrero et al. (Gene 138, 35-41 (1994) I.B.R.), Tsuchiya and Morinaga (Bio/Technology 6, 428-430 (1988) I.B.R.), in Eikmanns et al. (Gene 102, 93-98 (1991) I.B.R.), in European Patent Specification EPS 0 472 869 I.B.R., in U.S. Pat. No. 4,601,893 I.B.R., in Schwarzer and Pühler (Bio/Technology 9, 84-87 (1991) I.B.R., in Reinscheid et al. (Applied and Environmental Microbiology 60, 126-132 (1994) I.B.R.), in LaBarre et al. (Journal of Bacteriology 175, 1001-1007 (1993) I.B.R.), in Patent Application WO 96/15246 I.B.R., in Malumbres et al. (Gene 134, 15-24 (1993) I.B.R.), in Japanese Laid-Open Specification JP-A-10-229891 I.B.R., in Jensen and Hammer (Biotechnology and Bioengineering 58, 191-195 (1998) I.B.R.), in Makrides (Microbiological Reviews 60:512-538 (1996) I.B.R.) and in known textbooks of genetics and molecular biology.

By way of example, the pgsA2 gene according to the invention was over-expressed with the aid of plasmids.

Suitable plasmids are those which are replicated and expressed in coryneform bacteria. Numerous known plasmid vectors, such as e. g. pZ1 (Menkel et al., Applied and Environmental Microbiology (1989) 64: 549-554 I.B.R.), pEKEx1 (Eikmanns et al., Gene 102:93-98 (1991) I.B.R.) or pHS2-1 (Sonnen et al., Gene 107:69-74 (1991) I.B.R.) are based on the cryptic plasmids pHM1519, pBL1 or pGA1. Other plasmid vectors, such as e. g. those based on pCG4 (U.S. Pat. No. 4,489,160 I.B.R.), or pNG2 (Serwold-Davis et al., FEMS Microbiology Letters 66, 119-124 (1990) I.B.R.), or pAG1 (U.S. Pat. No. 5,158,891 I.B.R.), can be used in the same manner.

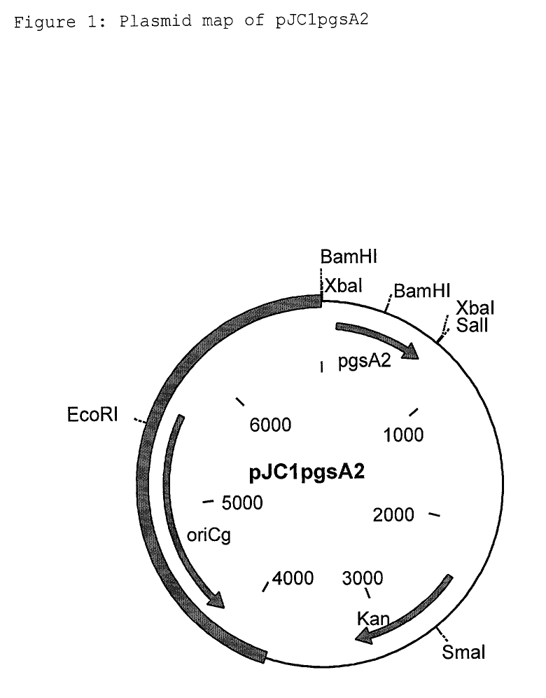

An example of a plasmid, with the aid of which the pgsA2 gene can be over-expressed is pJC1pgsA2 (FIG. 1), which is based on the E. coli -C. glutamicum shuttle vector pJC1 (Cremer et al., 1990, Molecular and General Genetics 220: 478-480) and contains the DNA sequence of C. glutamicum which codes for the pgsA2 gene. It is contained in the strain DSM5715/pJC1pgsA2.

Plasmid vectors which are moreover suitable are those with the aid of which the method of gene amplification by integration into the chromosome can be used, as has been described, for example, by Reinscheid et al. (Applied and Environmental Microbiology 60, 126-132 (1994) I.B.R.) for duplication or amplification of the homthrB operon. In this method, the complete gene is cloned in a plasmid vector which can replicate in a host (typically E. coli), but not in C. glutamicum. Possible vectors are, for example, pSUP301 (Simon et al., Bio/Technology 1, 784-791 (1983) I.B.R.), pK18mob or pK19mob (Schäfer et al., Gene 145, 69-73 (1994) I.B.R.), pGEM-T (Promega corporation, Madison, Wis., USA), pCR2.1-TOPO (Shuman (1994). Journal of Biological Chemistry 269:32678-84; U.S. Pat. No. 5,487,993) I.B.R., pCR®Blunt (Invitrogen, Groningen, Holland; Bernard et al., Journal of Molecular Biology, 234: 534-541 (1993) I.B.R.) or pEM1 (Schrumpf et al, 1991, Journal of Bacteriology 173:4510-4516 I.B.R.). The plasmid vector which contains the gene to be amplified is then transferred into the desired strain of C. glutamicum by conjugation or transformation. The method of conjugation is described, for example, by Schäfer et al. (Applied and Environmental Microbiology 60, 756-759 (1994) I.B.R.). Methods for transformation are described, for example, by Thierbach et al. (Applied Microbiology and Biotechnology 29, 356-362 (1988) I.B.R.), Dunican and Shivnan (Bio/Technology 7, 1067-1070 (1989) I.B.R.) and Tauch et al. (FEMS Microbiological Letters 123, 343-347 (1994) I.B.R.). After homologous recombination by means of a “cross over” event, the resulting strain contains at least two copies of the gene in question.

In addition, it may be advantageous for the production of amino acids, in particular L-lysine, to amplify or over-express one or more enzymes of the particular biosynthesis route, of glycolysis, of anaplerosis, of the citric acid cycle or of amino acid export, in addition to the pgsA2 gene.

Thus, for example, for the preparation of L-lysine, one or more genes chosen from the group consisting of

the dapA gene which codes for dihydrodipicolinate synthase (EP-B 0 197 335 I.B.R.), or

the dapE gene which codes for succinyl diaminopimelate desuccinylase, or

the lysC gene which codes for a feed-back resistant aspartate kinase (Kalinowski et al. (1990) I.B.R., Molecular and General Genetics 224, 317-324 I.B.R.), or

the gap gene which codes for glyceraldehyde 3-phosphate dehydrogenase (Eikmanns (1992), Journal of Bacteriology 174:6076-6086 I.B.R.), or

the tpi gene which codes for triose phosphate isomerase (Eikmanns (1992), Journal of Bacteriology 174:6076-6086 I.B.R.), or

the pgk gene which codes for 3-phosphoglycerate kinase (Eikmanns (1992), Journal of Bacteriology 174:6076-6086 I.B.R.), or

the pyc gene which codes for pyruvate carboxylase (DE-A-19831609 I.B.R.), or

the mqo gene which codes for malate-quinone oxidoreductase (Molenaar et al., European Journal of Biochemistry 254, 395-403 (1998) I.B.R.), or

the lysE gene which codes for lysine export (DE-A-195 48 222 I.B.R.)

can be amplified, in particular over-expressed or amplified, at the same time.

In addition to amplification of the pgsA2 gene it may moreover be advantageous for the production of amino acids, in particular L-lysine, to attenuate

the pck gene which codes for phosphoenol pyruvate carboxykinase (DE 199 50 409.1, DSM 13047 I.B.R.) and/or

the pgi gene which codes for glucose 6-phosphate isomerase (U.S. Ser. No. 09/396,478, DSM 12969 I.B.R.) and/or

the poxB gene which codes for pyruvate oxidase (DE: 1995 1975.7 I.B.R.)

at the same time.

In addition to over-expression of the pgsA2 gene it may moreover be advantageous for the production of amino acids, in particular L-lysine, to eliminate undesirable side reactions (Nakayama: “Breeding of Amino Acid Producing Micro-organisms”, in: Overproduction of Microbial Products, Krumphanzl, Sikyta, Vanek (eds.) I.B.R., Academic Press, London, UK, 1982).

The microorganisms prepared according to the invention can be cultured continuously or discontinuously in the batch method (batch culture) or in the fed batch (feed method) or repeated fed batch method (repetitive feed method) for the purpose of production of amino acids, in particular L-lysine. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991) I.B.R.) or in the textbook by Storhas (Bioreaktoren und periphere Einrichtungen [Bioreactors and Peripheral Equipment] (Vieweg Verlag, Braunschweig/Wiesbaden, 1994) I.B.R.).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981) I.B.R.. Sugars and carbohydrates, such as e. g. glucose, sucrose, lactose, fructose, maltose, molasses, starch and cellulose, oils and fats, such as e. g. soya oil, sunflower oil, groundnut oil and coconut fat, fatty acids, such as e. g. palmitic acid, stearic acid and linoleic acid, alcohols, such as e. g. glycerol and ethanol, and organic acids, such as e. g. acetic acid, can be used as the source of carbon. These substance can be used individually or as a mixture. Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture. Phosphoric acid, potassium dihydrogen phosphate or dipotassium hydrogen phosphate or the corresponding sodium-containing salts can be used as the source of phosphorus. The culture medium must moreover comprise salts of metals, such as e. g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH. Antifoams, such as e. g. fatty acid polyglycol esters, can be employed to control the development of foam. Suitable substances having a selective action, such as e. g. antibiotics, can be added to the medium to maintain the stability of plasmids. To maintain aerobic conditions, oxygen or oxygen-containing gas mixtures, such as e. g. air, are introduced into the culture. The temperature of the culture is usually 20° C. to 45° C., and preferably 25° C. to 40° C. Culturing is continued until a maximum of lysine has formed. This target is usually reached within 10 hours to 160 hours.

The analysis of L-lysine can be carried out by anion exchange chromatography with subsequent ninhydrin derivatization, as described by Spackman et al. (Analytical Chemistry, 30, (1958), 1190) I.B.R..

The following microorganism has been deposited at the Deutsche Sammlung für Mikrorganismen und Zellkulturen (DSMZ=German Collection of Microorganisms and Cell Cultures, Braunschweig, Germany) in accordance with the Budapest Treaty:

Corynebacterium glutamicum strain DSM5715/pJC1pgsA2 as DSM 13251

The method according to the invention is used for the fermentative preparation of amino acids, in particular L-lysine.

### EXAMPLES

The present invention is explained in more detail in the following with the aid of embodiment examples.

### Example 1

Preparation of a Genomic Cosmid Gene Library from Corynebacterium glutamicum ATCC 13032

Chromosomal DNA from Corynebacterium glutamicum ATCC 13032 was isolated as described by Tauch et al. (1995, Plasmid 33:168-179) I.B.R. and partly cleaved with the restriction enzyme Sau3AI (Amersham Pharmacia, Freiburg, Germany, Product Description Sau3AI, Code no. 27-0913-02) I.B.R. The DNA fragments were dephosphorylated with shrimp alkaline phosphatase (Roche Molecular Biochemicals, Mannheim, Germany, Product Description SAP, Code no. 1758250). The DNA of the cosmid vector SuperCos1 (Wahl et al. (1987) Proceedings of the National Academy of Sciences USA 84:2160-2164) I.B.R., obtained from Stratagene (La Jolla, USA, Product Description SuperCos1 Cosmid Vektor Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase. The cosmid DNA was then cleaved with the restriction enzyme BamHI (Amersham Pharmacia, Freiburg, Germany, Product Description BamHI, Code no. 27-0868-04). The cosmid DNA treated in this manner was mixed with the treated ATCC 13032 DNA gemischt and the batch was treated with T4 DNA ligase (Amersham Pharmacia, Freiburg, Germany, Product Description T4-DNA-Ligase, Code no. 27-0870-04). The ligation mixture was then packed in phages with the aid of Gigapack II XL Packing Extracts (Stratagene, La Jolla, USA, Product Description Gigapack II XL Packing Extract, Code no. 200217). For infection of the E. coli strain NM554 (Raleigh et al. 1988, Nucleic Acid Research 16:1563-1575 I.B.R.) the cells were taken up in 10 mM MgSO₄ and mixed with an aliquot of the phage suspension. The infection and titering of the cosmid library were carried out as described by Sambrook et al. (1989, Molecular Cloning: A laboratory Manual, Cold Spring Harbor I.B.R.), the cells being plated out on LB agar (Lennox, 1955, Virology, 1:190 I.B.R.) with 100 mg/l ampicillin. After incubation overnight at 37° C., recombinant individual clones were selected.

### Example 2

Isolation and Sequencing of the pgsA2 Gene

The cosmid DNA of an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany). The DNA of the sequencing vector pZero-1, obtained from Invitrogen (Groningen, Holland, Product Description Zero Background Cloning Kit, Product No. K2500-01) was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A laboratory Manual, Cold Spring Harbor) I.B.R., the DNA mixture being incubated overnight with T4 ligase (Pharmacia Biotech, Freiburg, Germany). This ligation mixture was then incorporated by means of electroporation (Tauch et al. 1994, FEMS Microbiol Letters, 123:343-7 I.B.R.) into the E. coli strain DH5αMCR (Grant, 1990, Proceedings of the National Academy of Sciences U.S.A., 87:4645-4649 I.B.R.) and plated out on LB agar (Lennox, 1955, Virology, 1:190 I.B.R.) with 50 mg/l zeocin. The plasmid preparation of the recombinant clones was carried out with Biorobot 9600 (Product No. 900200, Qiagen, Hilden, Germany). The sequencing was carried out by the dideoxy chain-stopping method of Sanger et al. (1977, Proceedings of the National Academy of Sciences U.S.A., 74:5463-5467) I.B.R. with modifications according to Zimmermann et al. (1990, Nucleic Acids Research, 18:1067) I.B.R. The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZero1 derivatives were assembled to a continuous contig. The computer-assisted coding region analysis was prepared with the XNIP program (Staden, 1986, Nucleic Acids Research, 14:217-231 I.B.R.). Further analyses were carried out with the “BLAST search program” (Altschul et al., 1997, Nucleic Acids Research, 25:3389-3402 I.B.R.), against the non-redundant databank of the “National Center for Biotechnology Information” (NCBI, Bethesda, Md., USA).

The resulting nucleotide sequence is shown in SEQ ID No. 1. Analysis of the nucleotide sequence showed an open reading frame of 291 base pairs, which was called the pgsA2 gene. The pgsA2 gene codes for a protein of 97 amino acids.

### Example 3

Cloning of the pgsA2 Gene in the Vector pJC1

Chromosomal DNA from Corynebacterium glutamicum ATCC 13032 was isolated as described by Tauch et al. (1995, Plasmid 33:168-179) I.B.R. A DNA fragment which carries the pgsA2 gene was amplified with the aid of the polymerase chain reaction. The following primers were used for this:

5′-TGC TCT AGA CGT CCG TCG AGA GGT TTT TAG G-3′
5′-TGC TCT AGA CCC CGC CAG ATT CTC CGA CAT -3′

Both oligonucleotides carry the sequence for the cleavage site of the restriction enzyme XbaI (nucleotides underlined). The primers shown were synthesized by MWG Biotech (Ebersberg, Germany) synthesized and the PCR reaction was carried out by the standard PCR method of Innis et al., (PCR protocol. A guide to methods and applications, 1990, Academic Press) I.B.R. The primers allow amplification of a DNA fragment of approx 749 bp in size, which carries the pgsA2 gene from Corynebacterium glutamicum.

After separation by gel electrophoresis, the PCR fragment was isolated from the agarose gel with the QiaExII Gel Extraction Kit (Product No. 20021, Qiagen, Hilden, Germany).

The PCR fragment obtained in this manner was cleaved completely with the restriction enzyme XbaI. The pgsA2 fragment approx 749 bp in size was isolated from the agarose gel with the QiaExII Gel Extraction Kit (Product No. 20021, Qiagen, Hilden, Germany).

The E. coli-C. glutamicum shuttle vector pJC1 (Cremer et al., 1990, Molecular and General Genetics 220: 478-480 I.B.R.) was used as the vector. This plasmid was also cleaved completely with the restriction enzyme XbaI and then dephosphorylated with shrimp alkaline phosphatase (Roche Molecular Biochemicals, Mannheim, Germany, Product Description SAP, Product No. 1758250).

The pgsA2 fragment obtained in this manner was mixed with the prepared vector pJC1 and the batch was treated with T4 DNA ligase (Amersham Pharmacia, Freiburg, Germany, Product Description T4-DNA-Ligase, Code no.27-0870-04). The ligation batch was transformed in the E. coli strain DH5α(Hanahan, In: DNA cloning. A practical approach. Vol. I. IRL-Press, Oxford, Washington D.C., USA) I.B.R.. Selection of plasmid-carrying cells was made by plating out the transformation batch on LB agar (Lennox, 1955, Virology, 1:190 I.B.R.) with 50 mg/l kanamycin. After incubation overnight at 37° C., recombinant individual clones were selected. Plasmid DNA was isolated from a transformant with the Qiaprep Spin Miniprep Kit (Product No. 27106, Qiagen, Hilden, Germany) in accordance with the manufacturer's instructions and cleaved with the restriction enzyme XbaI to check the plasmid by subsequent agarose gel electrophoresis. The resulting plasmid was called pJC1pgsA2.

### Example 4

Transformation of the Strain DSM5715 with the Plasmid pJC1pgsA2

The strain DSM5715 was transformed with the plasmid pJC1pgsA2 using the electroporation method described by Liebl et al., (FEMS Microbiology Letters, 53:299-303 (1989) I.B.R.). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl, 18 g/l Bacto-agar, which had been supplemented with 25 μg/ml kanamycin. Incubation was carried out for 2 days at 33° C.

Plasmid DNA was isolated from a transformant by conventional methods (Peters-Wendisch et al., 1998, Microbiology, 144, 915-927 I.B.R.) and cut with the restriction endonuclease EcoRI; in order to check the plasmid by subsequent agarose gel electrophoresis. The resulting strain was called DSM5715/pJC1pgsA2.

The strain DSM5715/pJC1pgsA2 has been deposited at the Deutsche Sammlung für Mikrorganismen und Zellkulturen (DSMZ=German Collection of Microorganisms and Cell Cultures, Braunschweig, Germany) in accordance with the Budapest Treaty as DSM 13251.

### Example 5

Preparation of Lysine

The C. glutamicum strain DSM5715/pJC1pgsA2 obtained in example 5 was cultured in a nutrient medium suitable for the production of lysine and the lysine content in the culture supernatant was determined.

For this, the strain was first incubated on an agar plate with the corresponding antibiotic (brain-heart agar with kanamycin (50 μg/ml) for 24 hours at 33° C. Starting from this agar plate culture, a preculture was seeded (10 ml medium in a 100 ml conical flask). The complete medium CgIII was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Kanamycin (25 mg/l) was added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

Medium MM

| | | |
|---|---|---|
| CSL (corn steep liquor) | 5 | g/l |
| MOPS (morpholinopropanesulfonic | 20 | g/l |
| acid) | | |
| Glucose (autoclaved separately) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state.

Culturing is carried out in a 10 ml volume in a 100 ml conical flask with baffles. Kanamycin (25 μg/ml) was added. Culturing was carried out at 33° C. and 80% atmospheric humidity.

After 24 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivatization with ninhydrin detection.

The result of the experiment is shown in table 1.

TABLE 1

| | | Lysine-HCl |
|---|---|---|
| Strain | OD(660) | g/l |
| DSM5715/pJC1pgSA2 | 12.1 | 8.60 |
| DSM5715 | 11.9 | 7.80 |

We claim:

1. A genetically modified coryneform bacterium, wherein the pgsA2 gene, which codes for CDP-diacylglycerol-glycerol-3-phosphate 3-phosphatidyltransferase, is amplified.

2. The genetically modified coryneform bacterium as claimed in claim 1, wherein the starting bacterium (wild-type) is selected from the group consisting of Corynebacterium glutamicum (ATCC13032), Corynebacterium acetoglutamicum (ATCC15806), Corynebacterium acetoacidophilum (ATCC13870), Corynebacterium thermoaminogenes (FERM BP-1539), Corynebacterium melassecola (ATCC17965), Brevibacterium flavum (ATCC14067), Brevibacterium lactofermentum (ATCC13869) and Brevibacterium divaricatum (ATCC14020), or is chosen from the group consisting of Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464 and Corynebacterium glutamicum DSM5715.

3. The genetically modified coryneform bacterium as claimed in claim 1, wherein the amplification of the pgsA2 gene is carried out by over-expression of the gene.

4. The genetically modified coryneform bacterium as claimed in claim 3, wherein the gene is amplified by increasing the number of copies of the gene, by choosing a potent promoter, by choosing a regulation region above the reading frame, by mutation of the promoter, by mutation of the regulation region, by mutation of the ribosome binding site, by incorporation of a suitable expression cassette above the structural gene, by incorporation of inducible promoters, by prolonging the life of the corresponding mRNA, by a reduced degradation of the proteins expressed, or by combination of several of these possibilities.

5. The genetically modified coryneform bacterium as claimed in claim 1, wherein the strain is transformed with a plasmid vector and the plasmid vector carries the nucleotide sequence which codes for the pgsA2 gene.

6. The genetically modified coryneform bacterium as claimed in claim 1, wherein it corresponds genotypically to the strain Corynebacterium glutamicum DSM 13251.

7. An isolated polynucleotide from coryneform bacteria, comprising a polynucleotide sequence selected from the group consisting of
a) a polynucleotide which is homologous to the extent of at least 70% to a polynucleotide which codes for a polypeptide which comprises the amino acid sequence of SEQ ID No. 2,
b) a polynucleotide which codes for a polypeptide which comprises an amino acid sequence which is homologous to the extent of at least 70% to the amino acid sequence of SEQ ID No. 2,
c) a polynucleotide which is complementary to the polynucleotides of a) or b), and
d) a polynucleotide comprising at least 15 successive nucleotides of the polynucleotide sequence of a), b) or c).

8. The polynucleotide as claimed in claim 7, wherein the polynucleotide is a recombinant DNA which is capable of replication in coryneform bacteria.

9. The polynucleotide as claimed in claim 7, wherein the polynucleotide is an RNA.

10. The polynucleotide as claimed in claim 8, wherein the DNA is capable of replication and comprises,
i) the nucleotide sequence shown in SEQ ID no. 1, or
ii) at least one sequence which corresponds to sequence (i) in the context of the degeneration of the genetic code, or
iii) at least one sequence which hybridizes with the sequence complementary to sequence(i) or (ii).

11. The polynucleotide as claimed in claim 10, wherein the DNA further comprises
iv) mutations of neutral function in (i) which lead to homologous amino acids.

12. The polynucleotide sequence as claimed in claim 8, which comprises DNA which codes for a polypeptide which has the amino acid sequence SEQ ID No. 2.

13. A method for the fermentative preparation of L-amino acids, comprising:
a) fermentating coryneform bacteria which produce L-amino acids and in which at least the pgsA2 gene or nucleotide sequences which code for it are amplified, in particular over-expressed.

14. The method as claimed in claim 13, further comprising:
b) concentration of the L-amino acid in the medium or in the cells of the bacteria.

15. The method as claimed in claim 14, further comprising:
c) isolation of the L-amino acid.

16. The method as claimed in claim 13, wherein a genetically modified coryneform bacterium, wherein the pgsA2 gene, which codes for CDP-diacylglycerol-glycerol-3-phosphate 3-phosphatidyltransferase, is amplified is employed.

17. The method as claimed in claim 13, wherein further genes which code for a protein of the biosynthesis pathway of the desired L-amino acid are additionally amplified in the bacteria.

18. The method as claimed in claim 13, wherein metabolic pathways which reduce the formation of the desired amino acid are at least partly eliminated in the bacteria.

19. The method as claimed in claim 13, wherein the amino acid prepared is L-lysine.

20. The method as claimed in claim 13, wherein for the preparation of lysine, bacteria in which at the same time one or more genes selected from the group consisting of
a) the dapA gene which codes for dihydrodipicolinate synthase,
b) the dapE gene which codes for succinyl diaminopimelate desuccinylase,
c) the lysC gene which codes for a feed-back resistant aspartate kinase,
d) the tpi gene which codes for triose phosphate isomerase,
e) the gap gene which codes for glyceraldehyde 3-phosphate dehydrogenase,
f) the pgk gene which codes for 3-phosphoglycerate kinase,
g) the pyc gene which codes for pyruvate carboxylase,
h) the mqo gene which codes for malate:quinone oxidoreductase, and
i) the lysE gene which codes for lysine export, is amplified and at the same time is or are fermented.

21. The method as claimed in claim 20, wherein said one or more genes is or are over expressed and at the same time is or are fermented.

22. The method as claimed in claim 13, wherein for the preparation of L-lysine, bacteria in which one or more genes selected from the group consisting of
a) the pck gene which codes for phosphoenol pyruvate carboxykinase,
b) the pgi gene which codes for glucose 6-phosphate isomerase, and
c) the poxB gene which codes for pyruvate oxidase, is attenuated at the same time are said bacteria fermented.

23. A primer which comprises a polynucleotide sequence sequences as claimed in claim 6 or parts thereof and can produce DNA of genes which code for CDP-diacylglycerol-glycerol-3-phosphate 3-phosphatidyltransferase by the polymer chain reaction.

24. A hybridization probe which comprises a polynucleotide sequences as claimed in claim 6 and can isolate cDNA or genes which have a high homology with the sequence of the pgsA2 gene.
